(19) United States
(12) Patent Application Publication
(10) Pub. No.: US 2007/0135406 A1
(43) Pub. Date: Jun. 14, 2007

(54) Diazabicyclononene and tetrahydropyridine derivatives with a new side-chain

(76) Inventors: Olivier Bezencon, Riehen (CH); Daniel Bur, Therwil (CH); Walter Fischli, Allschwil (CH); Lubos Remen, Allschwil (CH); Sylvia Richard-Bildstein, Dietwiller (FR); Thierry Sifferlen, Guewenheim (FR); Thomas Weller, Binningen (CH)

Correspondence Address: HOXIE & TSO LLP, 374 MILLBURN AVENUE, SUITE 300 E, MILLBURN, NJ, 07041, US

(21) Appl. No.: 10/581,824

(22) PCT Filed: Nov. 30, 2004
(86) PCT No.: PCT/EP04/13579

§ 371 Date: Jun. 2, 2006

(30) Foreign Application Priority Data
Dec. 5, 2003 (EP) PCT/EP03/13771

(51) Int. Cl. A61K 31/554; A61K 31/553; A61K 31/551; A61K 31/55

(52) U.S. Cl. 514/211.09; 514/214.03; 514/221; 540/549; 540/557; 540/581

(57) ABSTRACT

The invention relates to novel bicyclic derivatives, and related compounds and their use as active ingredients in the preparation of pharmaceutical compositions. The invention also concerns related aspects including processes for the preparation of the compounds, pharmaceutical compositions containing one or more of those compounds and especially their use as inhibitors of renin.

The invention relates to novel five-membered heteroaryl derivatives of the general formula (I). The invention also concerns related aspects including processes for the preparation of the compounds, pharmaceutical compositions containing one or more compounds of formula (I) and especially their use as renin inhibitors in cardiovascular events and renal insufficiency.

In the renin-angiotensin system (RAS) the biologically active angiotensin II (Ang II) is generated by a two-step mechanism. The highly specific enzyme renin cleaves angiotensinogen to angiotensin I (Ang I), which is then further processed to Ang II by the less specific angiotensin-converting enzyme (ACE). Ang II is known to work on at least two receptor subtypes called AT₁ and AT₂. Whereas AT₁ seems to transmit most of the known functions of Ang II, the role of AT₂ is still unknown.

Modulation of the RAS represents a major advance in the treatment of cardiovascular diseases. ACE inhibitors and AT₁ blockers have been accepted to treat hypertension (Waeber B. et al., “The renin-angiotensin system: role in experimental and human hypertension”, in Berkenhager W. H., Reid J. L. (eds): Hypertension, Amsterdam, Elsevier Science Publishing Co, 1996, 489-519; Weber M. A., Am. J. Hypertens., 1992, 5, 247S). In addition, ACE inhibitors are used for renal protection (Rosenberg M. E. et al., Kidney International, 1994, 45, 403; Breyer J. A. et al., Kidney International, 1994, 45, S156), in the prevention of congestive heart failure (Vaughan D. E. et al., Cardiovasc. Res., 1994, 28, 159; Fouad-Tarazi F. et al, Am. J. Med., 1988, 84 (Suppl. 3A), 83) and myocardial infarction (Pfeffer M. A. et al., N. Engl. J. Med., 1992, 327, 669).

The rationale to develop renin inhibitors is the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be by-passed by chymase, a serine protease (Husain A., J. Hypertens., 1993, 11, 1155). In patients inhibition of ACE thus leads to bradykinin accumulation causing cough (5-20%) and potentially life-threatening angioneurotic edema (0.1-0.2%) (Israili Z. H. et al., Annals of Internal Medicine, 1992, 117, 234). Chymase is not inhibited by ACE inhibitors. Therefore, the formation of Ang II is still possible in patients treated with ACE inhibitors. Blockade of the AT₁ receptor (e.g. by losartan) on the other hand overexposes other AT-receptor subtypes (e.g. AT₂) to Ang II, whose concentration is significantly increased by the blockade of AT₁ receptors. In summary, renin inhibitors are expected to demonstrate a different pharmaceutical profile than ACE inhibitors and AT₁ blockers with regard to efficacy in blocking the RAS and in safety aspects.

Only limited clinical experience (Azizi M. et al., J. Hypertens., 1994, 12, 419; Neutel J. M. et al., Am. Heart, 1991, 122, 1094) has been created with renin inhibitors because of their insufficient oral activity due to their peptidomimetic character (Kleinert H. D., Cardiovasc. Drugs, 1995, 9, 645). The clinical development of several compounds has been stopped because of this problem together with the high cost of goods. Only one compound containing four chiral centers has entered clinical trials (Rahuel J. et al., Chem. Biol., 2000, 7, 493; Mealy N. E., Drugs of the Future, 2001, 26, 1139). Thus, renin inhibitors with good oral bioavailability and long duration of action are required. Recently, the first non-peptide renin inhibitors were described which show high in vitro activity (Oefner C. et al., Chem. Biol., 1999, 6, 127; Patent Application WO97/09311; Mäurki H. P. et al., II Farmaco, 2001, 56, 21). However, the development status of these compounds is not known.

The present invention relates to the identification of renin inhibitors of a non-peptidic nature and of low molecular weight. Described are orally active renin inhibitors of long duration of action which are active in indications beyond blood pressure regulation where the tissular renin-chymase system may be activated leading to pathophysiologically altered local functions such as renal, cardiac and vascular remodeling, atherosclerosis, and possibly restenosis. So, the present invention describes these non-peptidic renin inhibitors.

In particular, the present invention relates to novel compounds of the general formula I, 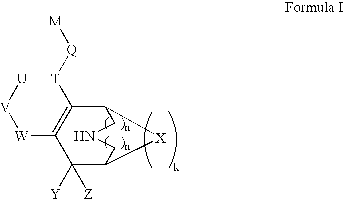
wherein
Y and Z represent independently from each other hydrogen, fluorine or a methyl group, or Y and Z may together form a cyclopropyl ring; in case k represents the integer 1, Y and Z both represent hydrogen;
X represents —(CH₂)ₘ—N(L)-(CH₂)ₘ—; —CH₂—CH(K)—CH₂—; —CH₂CH₂—; —CH₂OCH₂—; —CH₂SCH₂—; —CH₂SOCH₂—; —CH₂SO₂CH₂—; —CO—NL-CO—; —CO—NL-CHR⁶—; —CHR⁶—NL-CO—;
W represents a six-membered, non benzofused, phenyl or heteroaryl ring, substituted by V in position 3 or 4;
V represents a bond; —(CH₂)ᵣ—; -A-(CH₂)ₛ—; —CH₂-A-(CH₂)ₜ—; —(CH₂)ₛ-A-; —(CH₂)₂-A-(CH₂)ᵤ—; -A-(CH₂)ᵥ—B—; —CH₂—CH₂—CH₂-A-CH₂—; -A-CH₂—CH₂—B—CH₂—; —CH₂-A-CH₂—CH₂—B—; —CH₂—CH₂—CH₂-A-CH₂—CH₂—; —CH₂—CH₂—CH₂—CH₂-A-CH₂—; -A-CH₂—CH₂—B—CH₂—CH₂—; —CH₂-A-CH₂—CH₂—B—CH₂—; —CH₂-A-CH₂—CH₂—CH₂—B—; or —CH₂—CH₂-A-CH₂—CH₂—B—; —O—CH₂—CH(OCH₃)—CH₂—O; —O—CH₂—CH(CH₃)—CH₂—O—; —O—CH₂—CH(CF₃)—CH₂—O—; —O—CH₂—C(CH₃)₂—CH₂—O—; —O—CH₂—C(CH₃)₂—O—; —O—C(CH₃)₂—CH₂—O—; —O—CH₂—CH(CH₃)—O—; —O—CH(CH₃)—CH₂—O—; —O—CH₂—C(CH₂CH₂)—O—; —O—C(CH₂CH₂)—CH₂—O—;
A and B independently represent —O—; —S—; —SO—; —SO₂—;
U represents aryl; heteroaryl;
T represents —CONR¹—; —(CH₂)ₚOCO—; —(CH₂)ₚN(R¹)CO—; —(CH₂)ₚN(R¹)SO₂—; or —COO—;
Q represents lower alkylene; lower alkenylene;
M represents aryl-O(CH₂)ᵥR⁵; heteroaryl-O(CH₂)ᵥR⁵; aryl-O(CH₂)ᵥO(CH₂)_{w}R⁵; heteroaryl-(CH₂)ᵥO(CH₂)_{w}R⁵; aryl-OCH₂CH(R⁷)CH₂R⁵; heteroaryl-OCH₂CH(R⁷)CH₂R⁵;
L represents —R³; —COR³; —COOR³; —CONR²R³; —SO₂R³; —SO₂NR²R³;
—COCH(Aryl)₂;
K represents —H; —CH₂OR³; —CH₂NR²R³; —CH₂NR²COR³; —CH₂NR²SO₂R³; —CO₂R³; —CH₂OCONR²R³; —CONR²R³; —CH₂NR²CONR²R³; —CH₂SO₂NR²R³; —CH₂SR³; —CH₂SOR³; —CH₂SO₂R³;
R¹ represents hydrogen; lower alkyl; lower alkenyl; lower alkinyl; cycloalkyl; aryl; cycloalkyl-lower alkyl;
R² and R²′ independently represent hydrogen; lower alkyl; lower alkenyl; cycloalkyl; cycloalkyl-lower alkyl;
R³ represents hydrogen; lower alkyl; lower alkenyl; cycloalkyl; aryl; heteroaryl; heterocyclyl; cycloalkyl-lower alkyl; aryl-lower alkyl; heteroaryl-lower alkyl; heterocyclyl-lower alkyl; aryloxy-lower alkyl; heteroaryloxy-lower alkyl, whereby these groups may be unsubstituted or mono-, di- or trisubstituted with hydroxy, —OCOR², —COOR², lower alkoxy, cyano, —CONR²R²′, —CO-morpholin-4-yl, —CO-((4-loweralkyl)piperazin-1-yl), —NH(NH)NH₂, —NR⁴R⁴′ or lower alkyl, with the proviso that a carbon atom is attached at the most to one heteroatom in case this carbon atom is sp3-hybridized;
R⁴ and R⁴′ independently represent hydrogen; lower alkyl; cycloalkyl; cycloalkyl-lower alkyl; hydroxy-lower alkyl; —COOR²; —CONH₂;
R⁵ represents —OH, lower alkoxy, —OCOR², —COOR², —NR²R^{2′}, —OCONR²R²′, —NCONR²R²′, cyano, —CONR²R^{2′}, SO₃H, —SONR²R²′, —CO-morpholin-4-yl, —CO-((4-loweralkyl)piperazin-1-yl), —NH(NH)NH₂, —NR⁴R⁴′, with the proviso that a carbon atom is attached at the most to one heteroatom in case this carbon atom is sp3-hybridized;
R⁶ represents hydrogen; lower alkyl; lower alkoxy, whereby these groups may be unsubstituted or monosubstituted with hydroxy, —CONH₂, —COOH, imidazoyl, —NH₂, —CN, —NH(NH)NH₂;
R⁷ represents —OH, OR²; OCOR²; OCOR²; or R⁶ and R⁵ form together with the carbon atoms to which they are attached a 1,3-dioxolane ring which is substituted in position 2 with R² and R²′; or R⁶ and R⁵ form together with the carbon atoms to which they are attached a 1,3-dioxolan-2-one ring;
k is the integer 0 or 1;

m and n represent the integer 0 or 1, with the proviso that in case m represents the integer 1, n is the integer 0; in case n represents the integer 1, m is the integer 0; in case k represents the integer 0, n represents the integer 0; in case X does not represent —(CH₂)ₘ—N(L)-(CH₂)ₘ—, n represents the integer 0;

p is the integer 1, 2, 3 or 4;

r is the integer 1, 2, 3, 4, 5, or 6;

s is the integer 1, 2, 3, 4, or 5;

t is the integer 1, 2, 3, or 4;

u is the integer 1, 2, or 3;

v is the integer 1, 2, 3, or 4;

w is the integer 1 or 2.

In a preferred embodiment also the following forms are encompassed: optically pure enantiomers, mixtures of enantiomers such as racemates, diastereomers, mixtures of diastereomers, diastereomeric racemates, mixtures of diastereomeric racemates, and the meso-form; as well as pharmaceutically acceptable salts, solvent complexes and morphological forms.

In the definitions of general formula I—if not otherwise stated—the term lower alkyl, alone or in combination with other groups, means saturated, straight and branched chain groups with one to seven carbon atoms, preferably one to four carbon atoms that can be optionally substituted by halogens. Examples of lower alkyl groups are methyl, ethyl, n-propyl, iso-propyl, n-butyl, iso-butyl, sec-butyl, tert-butyl, pentyl, hexyl and heptyl. The methyl, ethyl and isopropyl groups are preferred.

The term lower alkoxy refers to a R—O group, wherein R is a lower alkyl. Examples of lower alkoxy groups are methoxy, ethoxy, propoxy, iso-propoxy, iso-butoxy, sec-butoxy and tert-butoxy.

For the substituent R⁸, the term lower alkoxy preferably refers to a methoxy group.

The term lower alkenyl, alone or in combination with other groups, means straight and branched chain groups comprising an olefinic bond and consisting of two to seven carbon atoms, preferably two to four carbon atoms, that can be optionally substituted by halogens.

Examples of lower alkenyl are vinyl, propenyl or butenyl.

The term lower alkinyl, alone or in combination with other groups, means straight and branched chain groups comprising a triple bond and consisting of two to seven carbon atoms, preferably two to four carbon atoms, that can be optionally substituted by halogens. Examples of lower alkinyl are ethinyl, propinyl or butinyl.

The term lower alkylene, alone or in combination with other groups, means straight and branched divalent chain groups with one to seven carbon atoms, preferably one to four carbon atoms, that can be optionally substituted by halogens. Examples of lower alkylene are methylene, ethylene, propylene or butylene.

For the substituent Q, the term lower alkylene preferably refers to a methylene group.

The term lower alkenylene, alone or in combination with other groups, means straight and branched divalent chain groups comprising an olefinic bond and consisting of two to seven carbon atoms, preferably two to four carbon atoms, that can be optionally substituted by halogens. Examples of lower alkenylene are vinylene, propenylene and butenylene.

The term lower alkylenedioxy, refers to a lower alkylene substituted at each end by an oxygen atom. Examples of lower alkylenedioxy groups are preferably methylenedioxy and ethylenedioxy.

The term lower alkylenoxy refers to a lower alkylene substituted at one end by an oxygen atom. Examples of lower alkylenoxy groups are preferably methylenoxy, ethylenoxy and propylenoxy.

The term halogen means fluorine, chlorine, bromine or iodine, preferably fluorine, chlorine and bromine.

The term cycloalkyl alone or in combination, means a saturated cyclic hydrocarbon ring system with 3 to 7 carbon atoms, e.g. cyclopropyl, cyclobutyl, cyclopentyl, cyclohexyl and cycloheptyl, which can be optionally mono- or multisubstituted by lower alkyl, lower alkenyl, lower alkenylene, lower alkoxy, lower alkylenoxy, lower alkylenedioxy, hydroxy, halogen, —CF₃, —NR¹R¹′, —NR¹C(O)R¹′, —NR¹S(O₂)R1′, —C(O)NR¹R¹′, lower alkylcarbonyl, —COOR¹, —SR¹, —SOR¹, —SO₂R¹, —SO₂NR¹R¹′ whereby R¹′ represents hydrogen; lower alkyl; lower alkenyl; lower alkinyl; cycloalkyl; aryl; cycloalkyl-lower alkyl. The cyclopropyl group is a preferred group.

The term aryl, alone or in combination, relates to the phenyl, the naphthyl or the indanyl group, preferably the phenyl group, which can be optionally mono- or multisubstituted by lower alkyl, lower alkenyl, lower alkinyl, lower alkenylene or lower alkylene forming with the aryl ring a five- or six-membered ring, lower alkoxy, lower alkylenedioxy, lower alkylenoxy, hydroxy, hydroxy-lower alkyl, halogen, cyano, —CF₃, —OCF₃, —NR¹R¹′, —NR¹R¹′-lower alkyl, —NR¹C(O)R¹′, —NR₁S(O₂)R¹, —C(O)NR¹R¹′, —NO₂, lower alkylcarbonyl, —COOR¹, —SR¹, —SOR¹, —SO₂R¹, —SO₂NR¹R¹′, benzyloxy, whereby R¹′ has the meaning given above. An example is 2-chloro-3,6-difluorophen-1-yl.

For the substituent U, the term aryl means preferably a mono-, di-, or trisubstituted phenyl whereby the substituents are halogen; lower alkyl or lower alkoxy. More preferred it means a mono-, di-, or trisubstituted phenyl whereby the substituents are selected from fluorine and chlorine. A preferred example is 2-chloro-3,6-difluorophen-1-yl.

The term aryloxy refers to an Ar—O group, wherein Ar is an aryl. An example of a lower aryloxy group is phenoxy.

The term heterocyclyl, alone or in combination, means saturated or unsaturated (but not aromatic) five-, six- or seven-membered rings containing one or two nitrogen, oxygen or sulfur atoms which may be the same or different and which rings can be optionally substituted with lower alkyl, hydroxy, lower alkoxy and halogen. The nitrogen atoms, if present, can be substituted by a —COOR² group. Examples of such rings are piperidinyl, morpholinyl, thiomorpholinyl, piperazinyl, tetrahydropyranyl, dihydropyranyl, 1,4-dioxanyl, pyrrolidinyl, tetrahydrofuranyl, dihydropyrrolyl, imidazolidinyl, dihydropyrazolyl, pyrazolidinyl, dihydroquinolinyl, tetrahydroquinolinyl, tetrahydroisoquinolinyl.

The term heteroaryl, alone or in combination, means six-membered aromatic rings containing one to four nitrogen atoms; benzofused six-membered aromatic rings containing one to three nitrogen atoms; five-membered aromatic rings containing one oxygen, one nitrogen or one sulfur atom; benzofused five-membered aromatic rings containing one oxygen, one nitrogen or one sulfur atom; five-membered aromatic rings containing one oxygen and one nitrogen atom and benzofused derivatives thereof; five-membered aromatic rings containing a sulfur and a nitrogen or an oxygen atom and benzofused derivatives thereof; five-membered aromatic rings containing two nitrogen atoms and benzofused derivatives thereof; five-membered aromatic rings containing three nitrogen atoms and benzofused derivatives thereof, or a tetrazolyl ring. Examples of such ring systems are furanyl, thiophenyl, pyrrolyl, pyridinyl, pyrimidinyl, indolyl, quinolinyl, isoquinolinyl, imidazolyl, triazinyl, thiazinyl, thiazolyl, isothiazolyl, pyridazinyl, pyrazolyl, oxazolyl, isoxazolyl, coumarinyl, benzothiophenyl, quinazolinyl, quinoxalinyl. Such rings may be adequately substituted with lower alkyl, lower alkenyl, lower alkinyl, lower alkylene, lower alkenylene, lower alkylenedioxy, lower alkyleneoxy, hydroxy-lower alkyl, lower alkoxy, hydroxy, halogen, cyano, —CF₃, —OCF₃, —NR¹R¹′, —NR¹R¹′-lower alkyl, —N(R¹)COR¹, —N(R¹)SO₂R¹, —CONR¹R¹′, —NO₂, lower alkylcarbonyl, —COOR¹, —SR¹, —SOR¹, —SO₂R¹, —SO₂NR¹R¹′, another aryl, another heteroaryl or another heterocyclyl and the like, whereby R¹′ has the meaning given above. An example is 3-methyl-pyridinyl, such as 3-methyl-pyridin-4-yl.

For the substituent M, the term heteroaryl means preferably a lower alkyl substituted pyridyl. More preferred it means 3-methyl-pyridinyl. A preferred example is 3-methyl-pyridin-4-yl.

The term heteroaryloxy refers to a Het-O group, wherein Het is heteroaryl as defined above.

The term cycloalkyl-lower alkyl refers to a cycloalkyl group which is substituted with a lower alkyl group as defined above.

The term aryl-lower alkyl refers to aryl group which is substituted with a lower alkyl group as defined above.

The term heteroaryl-lower alkyl refers to a heteroalkyl group which is substituted with a lower alkyl group as defined above.

The term heterocyclyl-lower alkyl refers to a heterocyclyl group which is substituted with a lower alkyl group as defined above.

The term aryloxy-lower alkyl refers to aryloxy group which is substituted with a lower alkyl group as defined above.

The term heteroaryloxy-lower alkyl refers to a heteroaryloxy group which is substituted with a lower alkyl group as defined above.

The term hydroxy-lower alkyl refers to a lower alkyl group which is substituted with a hydroxyl group.

The term lower alkylcarbonyl refers to a —CO-lower alkyl group.

The substituent maybe one of the following groups: aryl-O(CH₂)ᵥR; heteroaryl-O(CH₂)ᵥR⁸; aryl-O(CH₂)ᵥO(CH₂)_{w}R⁸; heteroaryl-(CH₂)ᵥO(CH₂)_{w}R⁸; aryl-OCH₂CH(R⁷)CH₂R⁵; heteroaryl-OCH₂CH(R⁷)CH₂R⁵. The aryl or heteroaryl groups are connected to the substituent Q. Examples for aryl are phenyl. Examples for heteroaryl are 3-methyl-pyridin-4-yl, and v is preferably 3. R⁸ is R⁵ as defined above; preferably R⁸ is lower alkoxy (most preferred methoxy). R⁵, R⁷ and w are as defined above.

The term sp3-hybridized refers to a carbon atom and means that this carbon atom forms four bonds to four substituents placed in a tetragonal fashion around this carbon atom.

The expression pharmaceutically acceptable salts encompasses either salts with inorganic acids or organic acids like hydrochloric or hydrobromic acid, sulfuric acid, phosphoric acid, citric acid, formic acid, acetic acid, maleic acid, tartaric acid, benzoic acid, methanesulfonic acid, p-toluenesulfonic acid, and the like that are non toxic to living organisms or in case the compound of formula I is acidic in nature with an inorganic base like an alkali or earth alkali base, e.g. sodium hydroxide, potassium hydroxide, calcium hydroxide and the like.

The compounds of the general formula I can contain two or more asymmetric carbon atoms and may be prepared in form of optically pure enantiomers, mixtures of enantiomers such as racemates, diastereomers, mixtures of diastereomers, diastereomeric racemates, mixtures of diastereomeric racemates, and the meso-form and pharmaceutically acceptable salts thereof.

The present invention encompasses all these forms. Mixtures may be separated in a manner known per se, i.e. by column chromatography, thin layer chromatography, HPLC or crystallization.

Another preferred embodiment of the invention are compounds of the general formula I, wherein

Y and Z represent independently from each other hydrogen, fluorine or a methyl group, or Y and Z may together form a cyclopropyl ring,

X represents —CH₂—CH(K)—CH₂—; —CH₂CH₂—; —CH₂OCH₂—; —CH₂SCH₂—; —CH₂SOCH₂—; —CH₂SO₂CH₂—; —CO—NL-CHR⁶—; —CHR⁶—NL-CO—;

W represents a six-membered, non benzofused, phenyl or heteroaryl ring, substituted by V in position 3 or 4;

V represents a bond; —(CH₂)ᵣ; -A-(CH₂)ₛ—; —CH₂-A-(CH₂)ₜ—; —(CH₂)ₛ-A-; —(CH₂)₂-A-(CH₂)ᵤ—; -A-(CH₂)ᵥ—B—; —CH₂—CH₂—CH₂-A-CH₂—; -A-CH₂—CH₂—B—CH₂—; —CH₂-A-CH₂—CH₂—B—; —CH₂—CH₂—CH₂-A-CH₂—CH₂—; —CH₂—CH₂—CH₂—CH₂-A-CH₂—; -A-CH₂—CH₂—B—CH₂—CH₂—; —CH₂-A-CH₂—CH₂—B—CH₂—; —CH₂-A-CH₂—CH₂—CH₂—B—; or —CH₂—CH₂-A-CH₂—CH₂—B—; —O—CH₂—CH(OCH₃)—CH₂—O; —O—CH₂—CH(CH₃)—CH₂—O—; —O—CH₂—CH(CF₃)—CH₂—O—; —O—CH₂—C(CH₃)₂—CH₂—O—; —O—CH₂—C(CH₃)₂—O—; —O—C(CH₃)₂—CH₂—O—; —O—CH₂—CH(CH₃)—O—; —O—CH(CH₃)—CH₂—O—; —O—CH₂—C(CH₂CH₂)—O—; —O—C(CH₂CH₂)—CH₂—O—;

A and B independently represent —O—; —S—; —SO—; —SO₂—;

U represents aryl; heteroaryl;

T represents —CONR¹—; —(CH₂)ₚOCO—; —(CH₂)ₚN(R¹)CO—; —(CH₂)ₚN(R¹)SO₂—; or —COO—;

Q represents lower alkylene; lower alkenylene;

M represents aryl-O(CH₂)ᵥR⁸; heteroaryl-O(CH₂)ᵥR⁸; aryl-O(CH₂)ᵥO(CH₂)_{w}R⁸; heteroaryl-(CH₂)ᵥO(CH₂)_{w}R⁸; aryl-OCH₂CH(R⁷)CH₂R⁵; heteroaryl-OCH₂CH(R⁷)CH₂R⁵;

L represents —R³; —COR³; —COOR³; —CONR²R³; —SO₂R³; —SO₂NR²R³;

—COCH(Aryl)₂;

K represents —H; —CH₂OR³; —CH₂NR²R³; —CH₂NR²COR³; —CH₂NR²SO₂R³; —CO₂R³; —CH₂OCONR²R³; —CONR²R³; —CH₂NR²CONR²R³; —CH₂SO₂NR²R³; —CH₂SR³; —CH₂SOR³; —CH₂SO₂R³;

R¹ represents hydrogen; lower alkyl; lower alkenyl; lower alkinyl; cycloalkyl; aryl; cycloalkyl-lower alkyl;

R² and R²′ independently represent hydrogen; lower alkyl; lower alkenyl; cycloalkyl; cycloalkyl-lower alkyl;

R³ represents hydrogen; lower allyl; lower alkenyl; cycloalkyl; aryl; heteroaryl; heterocyclyl; cycloalkyl-lower alkyl; aryl-lower alkyl; heteroaryl-lower alkyl; heterocyclyl-lower alkyl; aryloxy-lower alkyl; heteroaryloxy-lower alkyl, whereby these groups may be unsubstituted or mono-, di- or trisubstituted with hydroxy, —OCOR², —COOR², lower alkoxy, cyano, —CONR²R²′, —CO-morpholin-4-yl, —CO-((4-loweralkyl)piperazin-1-yl), —NH(NH)NH², —NR⁴R⁴′ or lower alkyl, with the proviso that a carbon atom is attached at the most to one heteroatom in case this carbon atom is sp3-hybridized;

R⁴ and R⁴′ independently represent hydrogen; lower alkyl; cycloalkyl; cycloalkyl-lower alkyl; hydroxy-lower alkyl; —COOR²; —CONH₂;

R⁵ represents —OH, lower alkoxy, —OCOR², —COOR², —NR²R^{2′}, —OCONR²R²′, —NCONR²R²′, cyano, —CONR²R²′, SO₃H, —SONR²R²′, —CO-morpholin-4-yl, —CO-((4-loweralkyl)piperazin-1-yl), —NH(NH)NH₂, —NR⁴R⁴′, with the proviso that a carbon atom is attached at the most to one heteroatom in case this carbon atom is sp3-hybridized;

R⁶ represents hydrogen; lower alkyl; lower alkoxy, whereby these groups may be unsubstituted or monosubstituted with hydroxy, —CONH₂, —COOH, imidazoyl, —NH₂, —CN, —NH(NH)NH₂;

R⁷ represents —OH, OR²; OCOR²; OCOOR²; or R⁶ and R⁵ form together with the carbon atoms to which they are attached a 1,3-dioxolane ring which is substituted in position 2 with R² and R²′; or R⁶ and R⁵ form together with the carbon atoms to which they are attached a 1,3-dioxolan-2-one ring;

R⁸ represents lower alkoxy,

p is the integer 1, 2, 3 or 4;

r is the integer 1, 2, 3, 4, 5, or 6;

s is the integer 1, 2, 3, 4, or 5;

t is the integer 1, 2, 3, or 4;

u is the integer 1, 2, or 3;

v is the integer 1, 2, 3, or 4;

w is the integer 1 or 2.

Another preferred embodiment of the invention are compounds of the general formula I, wherein X represents —CH₂CH₂—.

Another preferred embodiment of the invention are compounds of the general formula I, wherein

T represents —CONR¹—;

Q represents methylene;

M represents aryl-O(CH₂)ᵥR⁸; heteroaryl-O(CH₂)ᵥR⁸; aryl-OCH₂CH(R⁷)CH₂R⁵; heteroaryl-OCH₂CH(R⁷)CH₂R⁵.

Another preferred embodiment of the invention are compounds of the general formula I, wherein

R¹ represents cycloalkyl;

R⁸ represents lower alkoxy

v represents 3.

Another preferred embodiment of the invention are compounds of the general formula I as defined above, wherein W represents a 1,4-disubstituted pl.

Another preferred embodiment of the invention are compounds of the general formula I as defined above, wherein U is a mono-, di-, or trisubstituted phenyl whereby the substituents are halogen; lower alkyl or lower alkoxy.

Another preferred embodiment of the invention are compounds of the general formula I as defined above, wherein U is a mono-, di-, or trisubstituted phenyl whereby the substituents are selected from fluorine and chlorine.

Another preferred embodiment of the invention are compounds of the general formula I as defined above, wherein V represents -A-(CH₂)ₛ—.

Another preferred embodiment of the invention are compounds of the general formula I as defined above, wherein A represents —O—, and s represents 3.

Another preferred embodiment of the invention are compounds selected from the group consisting of

(rac.)-(1R *,5S*)-3-{4-[3-(2-chloro-3,6-difluorophenoxy)propyl]phenyl}-8-aza-bicyclo[3.2.1]oct-2-ene-2-carboxylic acid cyclopropyl-[2-(3-methoxypropoxy)-3-methylpyridin-4-ylmethyl]amide.

The invention relates to a method for the treatment and/or prophylaxis of diseases which are related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system, which method comprises administrating a compound as defined above to a human being or animal.

In another embodiment, the invention relates to a method for the treatment and/or prophylaxis of diseases which are related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, complications resulting from diabetes such as nephropathy, vasculopathy and neuropathy.

In another embodiment, the invention relates to a method for the treatment and/or prophylaxis of diseases, which are associated with a dysregulation of the renin-angiotensin system as well as for the treatment of the above-mentioned diseases.

The invention also relates to the use of compounds of formula (I) for the preparation of a medicament for the treatment and/or prophylaxis of the above-mentioned diseases.

A further aspect of the present invention is related to a pharmaceutical composition containing at least one compound according to general formula (I) and pharmaceutically acceptable carrier materials or adjuvants. This pharmaceutical composition may be used for the treatment or prophylaxis of the above-mentioned disorders; as well as for the preparation of a medicament for the treatment and/or prophylaxis of the above-mentioned diseases.

Derivatives of formula (I) or the above-mentioned pharmaceutical compositions are also of use in combination with other pharmacologically active compounds comprising ACE-inhibitors, neutral endopeptidase inhibitors, angiotensin II receptor antagonists, endothelin receptors antagonists, vasodilators, calcium antagonists, potassium activators, diuretics, sympatholitics, beta-adrenergic antagonists, alpha-adrenergic antagonists or with other drugs beneficial for the prevention or the treatment of the above-mentioned diseases.

In a preferred embodiment, this amount is comprised between 2 mg and 1000 mg per day.

In a particular preferred embodiment, this amount is comprised between 1 mg and 500 mg per day.

In a more particularly preferred embodiment, this amount is comprised between 5 mg and 200 mg per day.

All forms of prodrugs leading to an active component comprised by general formula (I) above are included in the present invention.

Compounds of formula (I) and their pharmaceutically acceptable acid addition salts can be used as medicaments, e.g. in the form of pharmaceutical compositions containing at least one compound of formula (I) and pharmaceutically acceptable inert carrier material or adjuvants. These pharmaceutical compositions can be used for enteral, parenteral, or topical administration. They can be administered, for example, perorally, e.g. in the form of tablets, coated tablets, dragees, hard and soft gelatine capsules, solutions, emulsions or suspensions, rectally, e.g. in the form of suppositories, parenterally, e.g. in the form of injection solutions or infusion solutions, or topically, e.g. in the form of ointments, creams or oils.

The production of pharmaceutical preparations can be effected in a manner which will be familiar to any person skilled in the art by bringing the described compounds of formula (I) and their pharmaceutically acceptable acid addition salts, optionally in combination with other therapeutically- valuable substances, into a galenical administration form together with suitable, non-toxic, inert, therapeutically compatible solid or liquid carrier materials and, if desired, usual pharmaceutical adjuvants.

Suitable carrier materials are not only inorganic carrier materials, but also organic carrier materials. Thus, for example, lactose, corn starch or derivatives thereof, talc, stearic acid or its salts can be used as carrier materials for tablets, coated tablets, dragees and hard gelatine capsules. Suitable carrier materials for soft gelatine capsules are, for example, vegetable oils, waxes, fats and semi-solid and liquid polyols (depending on the nature of the active ingredient no carriers are, however, required in the case of soft gelatine capsules). Suitable carrier materials for the production of solutions and syrups are, for example, water, polyols, sucrose, invert sugar and the like. Suitable carrier materials for injections are, for example, water, alcohols, polyols, glycerols and vegetable oils. Suitable carrier materials for suppositories are, for example, natural or hardened oils, waxes, fats and semi-liquid or liquid polyols. Suitable carrier materials for topical preparations are glycerides, semi-synthetic and synthetic glycerides, hydrogenated oils, liquid waxes, liquid paraffins, liquid fatty alcohols, sterols, polyethylene glycols and cellulose derivatives.

Usual stabilizers, preservatives, wetting and emulsifying agents, consistency-improving agents, flavour-improving agents, salts for varying the osmotic pressure, buffer substances, solubilizers, colorants and masking agents and antioxidants come into consideration as pharmaceutical adjuvants.

The dosage of compounds of formula (I) can vary within wide limits depending on the disease to be controlled, the age and the individual condition of the patient and the mode of administration, and will, of course, be fitted to the individual requirements in each particular case.

Another aspect of the invention is related to a process for the preparation of a pharmaceutical composition comprising a derivative of the general formula (I). According to said process, one or more active ingredients of the general formula (I) are mixing with inert excipients in a manner known per se.

The compounds of general formula I can be manufactured by the methods outlined below, by the methods described in the examples or by analogous methods.

Preparation of the Precursors:

Precursors are compounds which were prepared as key intermediates and/or building blocks and which were suitable for further transformations in parallel chemistry. Most of the chemistry applicable here has already been described in the patent applications WO03/093267 and WO04/002957.

As illustrated in Scheme 1 the known compound A can be derivatised into the corresponding triflate B. X¹ stands for a precursor of the substituent X as defined in general formula (I). The substituent X¹ can be transformed into the substituent X at any stage of the synthesis, whenever convenient. A Negishi-type coupling (or any other coupling catalysed by a transition metal) leads to a compound of type C, wherein R^{a} represents a precursor of the substituent U—V as defined in general formula (I). R^{a} can be easily transformed into U—V, using elemental chemical steps. After protecting group manipulation (→ compound of type D), ajustement of the W—V—U linker is possible for instance by deprotection and a Mitsunobu-type reaction, leading to a compound of type E. Hydrolysis of the ester leads to a carboxylic acid of type F, then an amide coupling for instance to a compound of type G. Removal of the Boc-protecting group and alkylation, or acylation, leads to a precursor of type H.

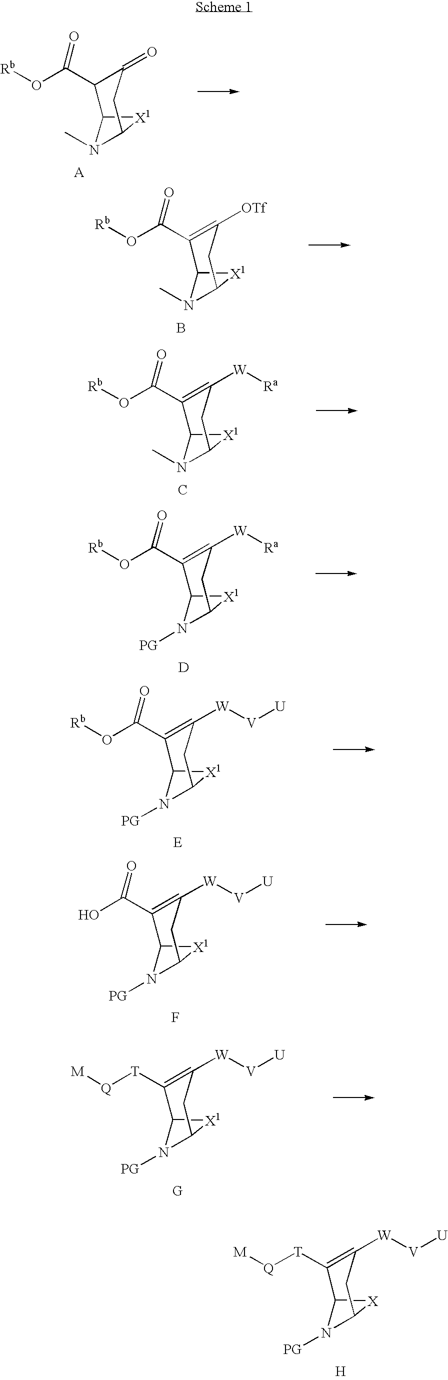

The bromoaryl components can be prepared as described in Scheme 2. A Mitsunobu coupling (→ compounds of type J) or the alkylation of an alcohol with a benzylic chloride (or bromide, → compounds of type K) are often the most convenient methods. Derivatives L and M were prepared in one step from 1-(3-chloropropoxymethyl)-2-methoxybenzene (Vieira E. et al., Bioorg. Med. Chem. Letters, 1999, 9, 1397) or 3-(5-bromopyridin-2-yloxy)propan-1-ol (Patent Application WO 98/39328) according to these methods. Other methods for the preparation of ethers or thioethers, like a Williamson synthesis, can be used as well (see e.g. March, J, “Advanced Organic Chemistry,”, 3^{rd} ed., John Wiley and sons, 1985). 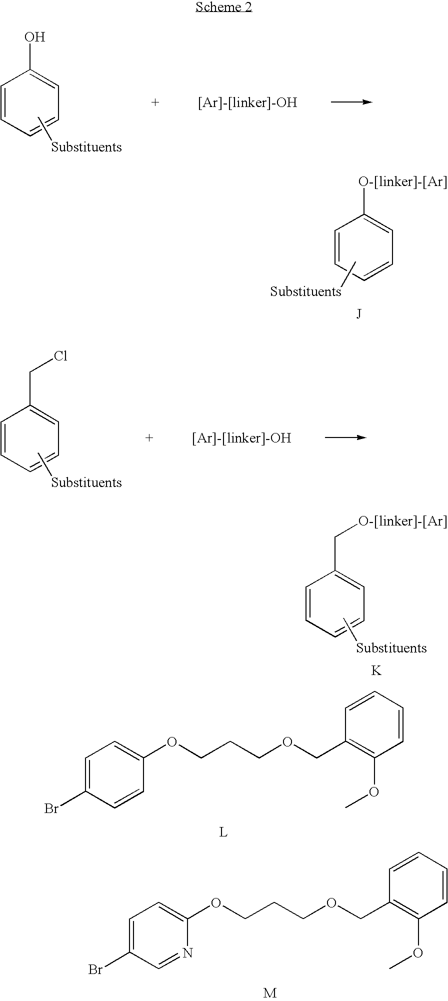
Preparation of the Secondary Amines

The secondary amines can be prepared for instance as described in Scheme 3. The pyridine derivative N can be prepared from commercially available 2-chloro-isonicotinoyl chloride. Deprotonation at the 3-position of this derivative, for instance with BuLi, and subsequent alkylation with a suitable electrophile leads to a derivative of type O, wherein R^{d} represents a suitable substituent that can be introduced by this chemistry, and can be transformed later into a desired substituent a described in general formula I. Reduction of the amide into an aldehyde with DIBAL leads to a compound of type P, then a reductive amination leads to an amine of type Q, wherein R¹ stand for a substituent as defined above. Finally substitution of the chlorine atom with an alcohol of type HO(CH2)ᵥR⁵, wherein R⁵ may still be protected, leads to an amine of type R. An alcohol of type HO(CH₂)₂O(CH₂)_{w}R⁵ can be introduced in the same way.

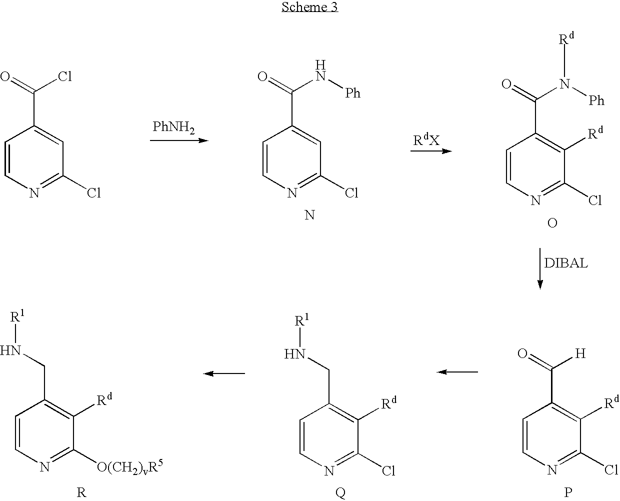

In the case of phenyl derivatives it is better to start from a compound of type S, wherein PG′ represents a suitable protecting group. Amide coupling with N-methylaniline leads to a derivative of type T, then deprotection to a derivative of type U. Ether bond formation, via a Mitsunobu-type reaction or from a corresponding alkyl halide, leads to a compound of type V. Reduction leads to an aldehyde of type W, then reductive amination to an amine of type X. An alcohol of type HO(CH₂)₂O(CH₂)_{w}R⁵ can be introduced in the same way. 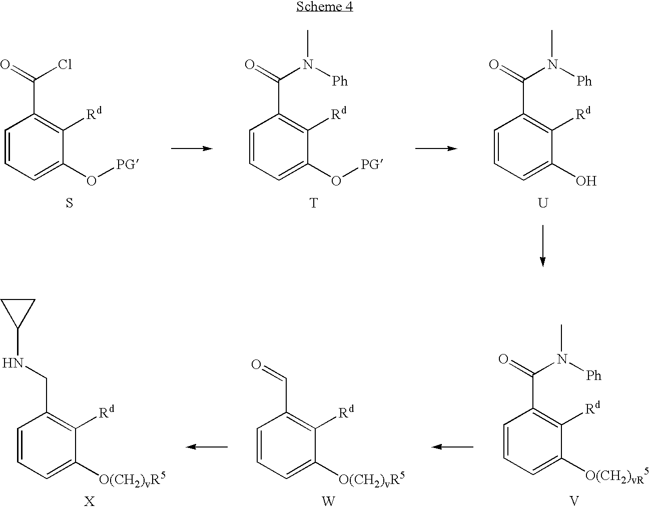
Preparation of Final Compounds

From precursors prepared as described above, the final compounds can be prepared using parallel chemistry techniques. For the specific examples, see the experimental part. Diazabicyclononenes of type of H can be deprotected using standard procedures (Scheme 5). Purification by preparative HPLC gives the corresponding TFA salts or formate salts.

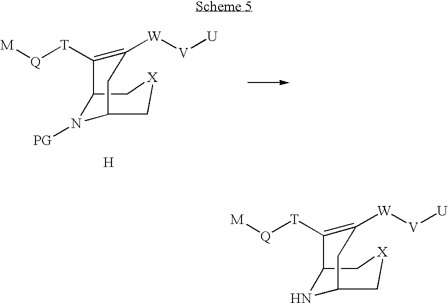

The following examples serve to illustrate the present invention in more detail. They are, however, not intended to limit its scope in any manner.

### EXAMPLES

Abbreviations

ACE Angiotensin Converting Enzyme
Ang Angiotensin
aq. aqueous
Boc tert-Butyloxycarbonyl
BSA Bovine serum albumine
BuLi n-Butyllithium
conc. concentrated
DIBAL Diisobutyl aluminium hydride
DIPEA Diisopropylethylamine
DMAP 4-N,N-Dimethylaminopyridine
DMF N,N-Dimethylformamide
DMSO Dimethylsulfoxide
EDCHCl Ethyl-N,N-dimethylaminopropylcarbodiimide hydrochloride
EIA Enzyme immunoassay
Et Ethyl
EtOAc Ethyl acetate
FC Flash Chromatography
HOBt Hydroxybenzotriazol
MeOH Methanol
org. organic
PG protecting group
RAS Renin Angiotensin System
rt room temperature
sat. saturated
sol. Solution
TBAF Tetra-n-butylammonium fluoride
TBDMS tert-Butyldimethylsilyl
Tf Trifluoromethylsulfonyl
THF Tetrahydrofuran

### (rac.)-(1R*,5S*)-8-Methyl-3-trifluoromethanesulfonyloxy-8-azabicyclo-[3.2.1]oct-2-ene-2-carboxylic acid methyl ester (B)

A sol. of compound 8-methyl-3-oxo-8-azabicyclo[3.2.1]octane-2-carboxylic acid methyl ester (Majewski, M., Lazny, R., J. Org. Chem., 1995, 60, 5825, 1.81 g, 9.12 mmol) in THF (35 mL) was cooled to 0° C. and NaH (about 60% in mineral oil, 435 mg, about 10.0 mmol) was added. A gas evolution was observed. After 20 min, Tf₂NPh (3.86 g, 10.8 mmol) was added. 10 min later, the ice bath was removed. The sol. was stirred overnight, and diluted with EtOAc and washed with brine (1×). The org. extracts were dried over MgSO₄, filtered, and the solvents were removed under reduced pressure. Purification by FC yielded the title compound (2.37 g, 78%).

### (rac.)-(1R*,5S*)-3-{4-[3-(tert-Butldimethylsilanyloxy)propyl]phenyl}-8-methyl-8-azabicyclo[3.2.1]oct-2-ene-2-carboxylic acid methyl ester (C)

A sol. of [3-(4-bromophenyl)propoxy]-tert-butyldimethylsilane (Kiesewetter D. O., Tetrahedron Asymmetry, 1993, 4, 2183, 16.47 g, 50.0 mmol) in THF (250 mL) was cooled to −78° C. BuLi (1.6M in hexane, 31.O mL, 50.0 mmol) was added. After 30 min, ZnCl₂ (1M in THF, 52 mL, 52 mmol, prepared from ZnCl₂ dried overnight at 150° C. and THF) was added. The mixture was allowed to warm up to rt. Vinyl triflate B (7.90 g, 24.0 mmol) in THF (20 mL) and then Pd(PPh₃)₄ (500 mg, 0.43 mmol) were added. The mixture was heated to reflux for 90 min and aq. 1M HCl (1 mL) was added. The mixture was diluted with EtOAc and washed with aq. 1M NaOH (1×). The org. extracts were dried over MgSO₄, filtered and the solvents were removed under reduced pressure. Purification of the residue by FC yielded the title product (8.44 g, 82%).

### (rac.)-(1R *,5S*)-3-[4-(3-Hydroxpropyl)phenyl]-8-azabicyclo[3.2.1]oct-2-ene-2,8-dicarboxylic acid 8-tert-butyl ester 2-methyl ester (D)

1- Chloroethyl chloroformate (7.98 g, 56.0 mmol) was added to a sol. of bicycloctene C (8.07 g, 18.8 mmol) in 1,2-dichloroethane (120 mL). The sol. was heated to reflux. After 4 h, the reaction mixture was allowed to cool to rt, and the solvents were removed under reduced pressure. MeOH (100 mL) was added. The mixture was stirred at 75° C. for 30 min, and the solvents were removed under reduced pressure. The residue was diluted with EtOAc and washed with aq. 1M NaOH (2×). The org. extracts were dried over MgSO₄, filtered, and the solvents were removed under reduced pressure. The residue was dissolved in CH₂Cl₂ (50 mL), DIPEA (4.70 g, 36.0 mmol) was added, and the mixture was cooled to 0° C. Boc₂O (4.65 g, 21.0 mmol) was added and the mixture was stirred at 0° C. for 1 h, then at rt for 2 h. The mixture was washed with aq. 1M HCl (1×), and aq. sat. NaHCO₃ (1×). The org. extracts were dried over MgSO₄, filtered, and the solvents were removed under reduced pressure. Purification of the residue by FC yielded the title compound (4.81 g, 64%).

### (rac.)-(1R*,5S*)-3-{4-[3-(2-Chloro-3,6-difluorophenoxy)propyl]phenyl}-8-azabicyclo[3.2.1]oct-2-ene-2,8-dicarboxylic acid 8-tert-butyl ester 2-methyl ester (E)

Tributylphosphine (1.61 mL, 7.2 mmol) was added to a sol. of bicycloctene D (1.04 g, 2.59 mmol), 2-chloro-3,6-trifluorophenol (833 mg, 5.10 mmol) and azodicarboxylic dipiperidide (1.29 g, 5.10 mmol) in toluene (25 mL). The mixture was heated to reflux for 2 h and allowed to cool to rt. The solvents were removed under reduced pressure. Purification by FC yielded the title compound (1.11 g, 78%).

### (rac-)-(1R*,5S*)-3-{4-[3-(2-Chloro-3,6-difluorophenoxy)propyl]phenyl}-8-azabicyclo[3.2.1]oct-2-ene-2,8-dicarboxylic acid 8-tert-butyl ester (F)

Bicycloctene E (2.42 g, 4.40 mmol) was dissolved in EtOH (50 mL). Aq. 1M NaOH (40 mL) was added and the mixture was heated to 80° C. The sol. was stirred for 5 h at 80° C., then allowed to cool down to rt. After acidification to pH =1-2 with aq. 1M HCl the mixture was extracted with EtOAc (3×). The combined org. extracts were dried over MgSO₄, filtered and the solvents were removed under reduced pressure. Purification of the residue by FC yielded the title compound (2.48 g, quantitative).

### (rac.)-(1R*,5S*)-2-({2-[3-(tert-Butldimethylsilanyloxy)propoxy]-3-methyl-pyridin-4-ylmethyl}cyclopropylcarbamoyl)-3-{4-[3-(2-chloro-3,6-difluoro-phenoxy)propyl]phenyl}-8-azabicyclo[3.2.1]oct-2-ene-8-carboxylic acid tert-butyl ester (G1)

To a sol. of compound F (3.45 g, 6.46 mmol) in CH₂Cl₂ (60 mL) were added the amine R (2.26 g, 6.46 mmol), DMAP (197 mg, 1.62 mmol), DIPEA (4.42 mL, 25.8 mmol), HOBt (1.30 g, 9.69 mmol), and EDC.HCl (3.09 g, 16.2 mmol). The mixture was stirred at rt overnight. EDC.HCl (2.00 g, 1.00 mmol) and DIPEA (3.50 mL, 20.4 mmol) were added. The mixture was stirred at rt for 3 days. Amine R (2.00 g, 5.71 mmol), EDC.HCl (2.00 g, 1.01 mmol), and HOBt (1.00 g, 7.40 mol) were added. After 2 days (total 6 days) the mixture was diluted with more CH₂Cl₂, washed with aq. 1M HCl (3×), and with aq. sat NaHCO₃ (1×). The org. extracts were dried over MgSO₄, filtered, and the solvents were removed under reduced pressure. Purification of the residue by FC (EtOAc/heptane 1:4→3:7→2:4) yielded the title compound (3.43 g, 61%).

### (rac.)-(1R*,5S*)-3-{4-[3-(2-Chloro-3,6-difluorophenoxy)propyl]phenyl}-8-azabicyclo[3.2.1]oct-2-ene-2-carboxylic acid cyclopropyl-[2-(3-hydroxy-propoxy)-3-methylpyridin-4-ylmethyl]amide (G2)

A sol. of compound G1 (3.43 g, 3.95 mmol) in CH₂Cl₂ (35 mL) was cooled to 0° C. HCl/dioxane (4M, 35 mL) was added. After 15 min the ice bath was removed and the mixture was stirred for 1 h at rt. The solvents were rapidly removed under reduced pressure and the residue was dried under high vacuum for 15 min. The residue was then diluted with CH₂Cl₂ and washed with aq. 1M NaOH (1×). The org. extracts were dried over MgSO₄, filtered, and the solvents were removed under reduced pressure. Purification of the residue by FC (MeOH/CH₂Cl₂ 5%→10%→15%→20%) yielded the title compound (1.25 g, 48%).

### (rac.)-(1R*,5S*)-3-{4-[3-(2-Chloro-3,6-difluorophenoxy)propyl]phenyl}-2-{cyclopropyl-[2-(3-hydroxypropoxy)-3-methylpyridin-4-ylmethyl]-carbamoyl}-8-azabicyclo[3.2.1]oct-2-ene-8-carboxylic acid tert-butyl ester (G3)

To a sol. of compound G2 (1.19 g, 1.82 mmol) in CH₂Cl₂ (5 mL) was added at 0° C. DIPEA (0.80 mL, 4.56 mmol) and Boc₂O (0.61 g 2.74 mmol). The mixture was stirred at 0° C. for 30 min and was concentrated under reduced pressure . Aq. sat. NH₄Cl (5 mL) was added and the mixture was extracted with CH₂Cl₂ (3×),. The org. extracts were dried over MgSO₄, filtered, and the solvents were removed under reduced pressure. Purification of the residue by FC (EtOAc/heptane=1:1) yielded the title compound (1.09 g, 80%).

### (rac.)-(1R*,5S*)-3-{4-[3-(2-Chloro-3,6-difluorophenoxy)propyl]phenyl}-2-{cyclopropyl-[2-(3-methoxypropoxy)-3-methylpyridin-4-ylmethyl]-carbamoyl}-8-azabicyclo[3.2.1]oct-2-ene-8-carboxylic acid tert-butyl ester (G4)

To a sol. of compound G3 (100 mg, 0.133 mmol) in DMF (2 mL) was added NaH (60% in oil, 96 mg, 0.22 mmol) at 0° C. under N₂, and the mixture was stirred for 1 h. MeI (22 μL, 0.133 mmol) was added and the mixture was stirred overnight. Water was added, and the mixture was extracted with CH₂Cl₂ (3×), dried over MgSO₄, filtered, and concentrated under reduced pressure. Purification of the residue by FC (EtOAc/heptane 1:1) yielded the title compound (102 mg, 49%).

### 2-Chloro-N-phenylisonicotinamide (N)

To the sol. of 2-chloro-isonicotinoyl chloride (Anderson, W. K., Dean, D.C., Endo, T., J. Med. Chem., 1990, 33, 1667, 10 g, 56.8 mmol) in 1,2-dichloroethane (100 mL) was added at 0° C. a sol. of aniline (5.70 mL, 62.5 mmol) and DIPEA (10.2 ml, 59.6 mmol) in 1,2-dichloroethane (10 ml) during ca. 30 ml. The reaction was stirred at 0° C. for ca. 30 min and subsequently for 1 h at 95° C. Water (30 mL) was added at rt and the mixture was filtered-off. The filtrate was extracted with CH₂Cl₂ (200 mL). The combined org. extracts were dried over MgSO₄, filtered, and the solvents were removed under reduced pressure. The residue was crystallized from MeOH/water 1:10 (110 mL), yielding the title compound (12.12 g, 92%). LC-MS: R_{T}=0.87 min; ES⁺=233.1.

### 2-Chloro-3-N-dimethyl-N-phenylisonicotinamide (O)

To a sol. of compound N (8.79 g, 37.8 mmol) in THF (90 mL) was added BuLi (1.6M in hexane, 52 mL, 83.2 mmol) at −78° C. After 30 min MeI (7.70 mL, 124 mmol) was added dropwise at the same temperature. The mixture was stirred at −78° C. for 1 h, and was warmed up to 33° C. The mixture was stirred at 33° C. for 30 min. Aq. 10% NH₄OH was added dropwise at rt, and the mixture was extracted with Et₂O. The org. extracts were dried over MgSO₄, filtered, and the solvents were evaporated under reduced pressure. Purification by FC yielded the title compound (8.67 g, 88%). LC-MS:R_{T}=0.85 min; ES⁺=261.2.

### 2-Chloro-3-methylpyridine-4-carbaldehyde (P)

To the sol. of pyridine derivative O (9.58 g, 36.7 mmol) in CH₂Cl₂ (190 mL) was at −78° C. added DIBAL (1M in CH₂Cl₂, 55.1 mL, 55.1 mmol), and the mixture was stirred at −78° C. for 1.5 h. Aq. sat. tartaric acid monosodium monokalium salt in water (20 ml) was added and the mixture was allowed to warm up to rt. Water was added and the mixture was extracted with CH₂Cl₂. The org. extracts were dried over MgSO₄, filtered, and the solvents were removed under reduced pressure. Purification of the residue by FC yielded the title compound (4.4 g, 77%). LC-MS:R_{T}=0.76 min; ES⁺=156.1.

### (2-Chloro-3-methylpyridin-4-ylmethyl)-cyclopropylamine (Q)

A sol. of aldehyde P (4.70 g, 30.2 mmol) and cyclopropylamine (4.20 ml, 60.4 mmol) in MeOH (65 mL) was stirred at rt for 4 h. NaBH₄ (1.55 g, 39.2 mmol) was added and the mixture was stirred at rt for 12 h. Water and subsequently aq. 1M NaOH were added, and the solvents were partially removed under reduced pressure. The water phase was extracted with CH₂Cl₂ (2×). The combined org. extracts were dried over MgSO₄, filtered, and the solvents were removed under reduced pressure. Purification of the crude by FC yielded the title compound (4.66 g, 79%). LC-MS:R_{T}=0.43 min; ES⁺=197.1.

### {2-[3-(tert-Butyldimethylsilanyloxy)propoxy]-3-methylpyridin-4-ylmethyl}-cyclopropylamine (R)

A sol. of amine Q (1.24 g, 6.30 mmol) and 2-(tert-butyldimethylsilanyloxy)-propan-1-ol (403 mg, 10.1 mmol) in dioxan (5 ml) was heated at 115° C. for 12 h. The solvents were removed under reduced pressure, water was added, and the mixture was extracted with Et₂O (2×). The combined org. extracts were dried over MgSO₄, filtered, and the solvents were removed under reduced pressure. Purification of the crude by FC yielded the title compound (192 mg, 9%). LC-MS:R_{T}=0.84 min; ES⁺=351.4.

### Example 1

### (rac.)-(1R*,5S*)-3-{4-[3-(2-Chloro-3,6-difluorophenoxy)propyl]phenyl}-8-azabicyclo[3.2.1]oct-2-ene-2-carboxylic acid cyclopropyl-[2-(3-methoxy-propoxy)-3-methylpyridin-4-ylmethyl]amide

A sol. of compound G4 (50 mg) was stirred under N₂ in a sol. HCl in Et₂O (2M, 2 mL) at rt overnight. The reaction mixture was concentrated and crude product was purified by FC (CH₂Cl₂/MeOH=9/1), which yielded the title compound (23 mg, 53%).

The following assay was carried out in order to determine the activity of the compounds of general formula I and their salts.

Inhibition of Human Recombinant Renin by the Compounds of the Invention

The enzymatic in vitro assay was performed in 384well polypropylene plates (Nunc). The assay buffer consisted of 10 mM PBS (Gibco BRL) including 1 mM EDTA and 0.1% BSA. The incubates were composed of 50 μL per well of an enzyme mix and 2.5 μL of renin inhibitors in DMSO. The enzyme mix was premixed at 4° C. and consists of the following components:
human recombinant renin (0.16 ng/mL).synthetic human angiotensin(1-14) (0.5 μM)
hydroxyquinoline sulfate (1 mM)

The mixtures were then incubated at 37° C. for 3 h.

To determine the enzymatic activity and its inhibition, the accumulated Ang I was detected by an enzyme immunoassay (EIA) in 384well plates (Nunc). 5 μL of the incubates or standards were transferred to immuno plates which were previously coated with a covalent complex of Ang I and bovine serum albumin (Ang I—BSA). 75 mL of Ang I-antibodies in essaybuffer above including 0.01% Tween 20 were added and a primary incubation made at 4° C. overnight The plates were washed 3 times with PBS including 0.01% Tween 20, and then incubated for 2 h at rt with an antirabbit-peroxidase coupled antibody (WA 934, Amersham). After washing the plates 3 times, the peroxidase substrate ABTS (2.2′-azino-di-(3-ethyl-benzthiazolinsulfonate), was added and the plates incubated for 60 min at room temperature. After stopping the reaction with 0.1 M citric acid pH 4.3 the plate was evaluated in a microplate reader at 405 nm. The percentage of inhibition was calculated of each concentration point and the concentration of renin inhibition was determined that inhibited the enzyme activity by 50% (IC₅₀). The IC₅₀-values of all compounds tested are below 10 μM.

Examples of Inhibition:

Example 1: 0.79 nM

1. Compounds of the general formula I 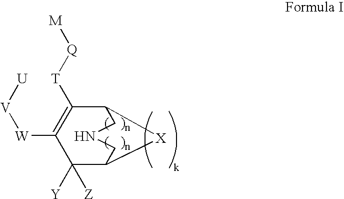
wherein
Y and Z represent independently from each other hydrogen, fluorine or a methyl group, or Y and Z may together form a cyclopropyl ring; in case k represents the integer 1, Y and Z both represent hydrogen;
X represents —(CH₂)ₘ—N(L)-(CH₂)ₘ—; —CH₂—CH(K)—CH₂—; —CH₂CH₂—; —CH₂OCH₂—; —CH₂SCH₂—; —CH₂SOCH₂—; —CH₂SO₂CH₂—; —CO—NL-CO—; —CO—NL-CHR⁶—; —CHR⁶—NL-CO—;
W represents a six-membered, non benzofused, phenyl or heteroaryl ring, substituted by V in position 3 or 4;
V represents a bond; —(CH₂)ᵣ—; -A-(CH₂)ᵣ—; —CH₂-A-(CH₂)ₜ—; —(CH₂)ₛ-A-; —(CH₂)₂-A-(CH₂)ᵤ—; -A-(CH₂)ᵥ—B—; —CH₂—CH₂—CH₂-A-CH₂—; -A-CH₂—CH₂—B—CH₂—; —CH₂-A-CH₂—CH₂—B—; —CH₂—CH₂—CH₂-A-CH₂—CH₂—; —CH₂—CH₂—CH₂—CH₂-A-CH₂—; -A-CH₂—CH₂—B—CH₂—CH₂—; —CH₂-A-CH₂—CH₂—B—CH₂—; —CH₂-A-CH₂—CH₂—CH₂—B—; or —CH₂—CH₂-A-CH₂—CH₂—B—; —O—CH₂—CH(OCH₃)—CH₂—O; —O—CH₂—CH(CH₃)—CH₂—O—; —O—CH₂—CH(CF₃)—CH₂—O—; —O—CH₂—C(CH₃)₂—CH₂—O—; —O—CH₂—C(CH₃)₂—O—; —O—C(CH₃)₂—CH₂—O—; —O—CH₂—CH(CH₃)—O—; —O—CH(CH₃)—CH₂—O—; —O—CH₂—C(CH₂CH₂)—O—; —O—C(CH₂CH₂)—CH₂—O—;
A and B independently represent —O—; —S—; —SO—; —SO₂—;
U represents aryl; heteroaryl;
T represents —CONR¹—; —(CH₂)ₚOCO—; —(CH₂)ₚN(R¹)CO—; —(CH₂)ₚN(R¹)SO₂—; or —COO—;
Q represents lower alkylene; lower alkenylene;
M represents aryl-O(CH₂)ᵥR⁵; heteroaryl-O(CH₂)ᵥR⁵; aryl-O(CH₂)ᵥO(CH₂)_{w}R⁵; heteroaryl-(CH₂)ᵥO(CH₂)_{w}R⁵; aryl-OCH₂CH(R⁷)CH₂R⁵; heteroaryl-OCH₂CH(R⁷)CH₂R⁵;
L represents —R³; —COR³; —COOR³; —CONR²R³; —SO₂R³; —SO₂NR²R³;
—COCH(Aryl)₂;
K represents —H; —CH₂OR³; —CH₂NR²R³; —CH₂NR²COR³; —CH₂NR²SO₂R³; —CO₂R³; —CH₂OCONR²R³; —CONR²R³; —CH₂NR²CONR²R³; —CH₂SO₂NR²R³; —CH₂SR³; —CH₂SOR³; —CH₂SO₂R³;
R¹ represents hydrogen; lower alkyl; lower alkenyl; lower alkinyl; cycloalkyl; aryl; cycloalkyl-lower alkyl;
R² and R²′ independently represent hydrogen; lower alkyl; lower alkenyl; cycloalkyl; cycloalkyl-lower alkyl;
R³ represents hydrogen; lower alkyl; lower alkenyl; cycloalkyl; aryl; heteroaryl; heterocyclyl; cycloalkyl-lower alkyl; aryl-lower alkyl; heteroaryl-lower alkyl; heterocyclyl-lower alkyl; aryloxy-lower alkyl; heteroaryloxy-lower alkyl, whereby these groups may be unsubstituted or mono-, di- or trisubstituted with hydroxy, —OCOR², —COOR², lower alkoxy, cyano, —CONR²R²′, —CO-morpholin-4-yl, —CO-((4-loweralkyl)piperazin-1-yl), —NH(NH)NH₂, —NR⁴R⁴′ or lower alkyl, with the proviso that a carbon atom is attached at the most to one heteroatom in case this carbon atom is sp3-hybridized;
R⁴ and R⁴′ independently represent hydrogen; lower alkyl; cycloalkyl; cycloalkyl-lower alkyl; hydroxy-lower alkyl; —COOR²; —CONH₂;
R⁵ represents —OH, lower alkoxy, —OCOR², —COOR², —NR²R^{2′}, —OCONR²R²′, —NCONR²R²′, cyano, —CONR²R²′, SO₃H, —SONR²R²′, —CO-morpholin-4-yl, —CO-((4-loweralkyl)piperazin-1-yl), —NH(NH)NH², —NR⁴R⁴′, with the proviso that a carbon atom is attached at the most to one heteroatom in case this carbon atom is sp3-hybridized;
R⁶ represents hydrogen; lower alkyl; lower alkoxy, whereby these groups may be unsubstituted or monosubstituted with hydroxy, —CONH₂,
—COOH, imidazoyl, —NH₂, —CN, —NH(NH)NH₂;
R⁷ represents —OH, OR²; OCOR²; OCOOR²; or R⁶ and R⁵ form together with the carbon atoms to which they are attached a 1,3-dioxolane ring which is substituted in position 2 with R² and R²′;
or R⁶ and R⁵ form together with the carbon atoms to which they are attached a 1,3-dioxolan-2-one ring;
k is the integer 0 or 1;
m and n represent the integer 0 or 1, with the proviso that in case m represents the integer 1, n is the integer 0; in case n represents the integer l, m is the integer 0; in case k represents the integer 0, n represents the integer 0; in case X does not represent —(CH₂)ₘ—N(L)-(CH₂)ₘ—, n represents the integer 0;
p is the integer 1, 2, 3 or 4;
r is the integer 1, 2, 3, 4, 5, or 6;
s is the integer 1, 2, 3, 4, or 5;
t is the integer 1, 2, 3, or 4,
u is the integer 1, 2, or 3;
v is the integer 1, 2, 3, or 4;
w is the integer 1 or 2;
in any form, including optically pure enantiomers, mixtures of enantiomers such as racemates, diastereomers, mixtures of diastereomers, diastereomeric racemates, mixtures of diastereomeric racemates, and the meso-form; as well as free or pharmaceutically acceptable salts, solvent complexes and morphological forms.

2. Compounds according to claim 1, wherein
Y and Z represent independently from each other hydrogen, fluorine or a methyl group, or Y and Z may together form a cyclopropyl ring;
X represents —CH₂—CH(K)—CH₂—; —CH₂CH₂—; —CH₂OCH₂—; —CH₂SCH₂—; —CH₂SOCH₂—; —CH₂SO₂CH₂—; —CO—NL-CHR⁶—; —CHR⁶—NL-CO—;
W represents a six-membered, non benzofused, phenyl or heteroaryl ring, substituted by V in position 3 or 4;
V represents a bond; —(CH₂)ᵣ—; -A-(CH₂)ₛ—; —CH₂-A-(CH₂)ₜ—; —(CH₂)ₛ-A-; —(CH₂)₂-A-(CH₂)ᵤ—; -A-(CH₂)ᵥ—B—; —CH₂—CH₂—CH₂-A-CH₂—; -A-CH₂—CH₂—B—CH₂—; —CH₂-A-CH₂—CH₂—B—; —CH₂—CH₂—CH₂-A-CH₂—CH₂—; —CH₂—CH₂—CH₂—CH₂-A-CH₂—; -A-CH₂—CH₂—B—CH₂—CH₂—; —CH₂-A-CH₂—CH₂—B—CH₂—; —CH₂-A-CH₂—CH₂—CH₂—B—; or —CH₂—CH₂-A-CH₂—CH₂—B—; —O—CH₂—CH(OCH₃)—CH₂—O; —O—CH₂—CH(CH₃)—CH₂—O—; —O—CH₂—CH(CF₃)—CH₂—O—; —O—CH₂—C(CH₃)₂—CH₂—O—; —O—CH₂—C(CH₃)₂—O—; —O—C(CH₃)₂—CH₂—O—; —O—CH₂—CH(CH₃)—O—; —O—CH(CH₃)—CH₂—O—; —O—CH₂—C(CH₂CH₂)—O—; —O—C(CH₂CH₂)—CH₂—O—;
A and B independently represent —O—; —S—; —SO—; —SO₂—;
U represents aryl; heteroaryl;
T represents —CONR¹—; —(CH₂)ₚOCO—; —(CH₂)ₚN(R¹)CO—; —(CH₂)ₚN(R¹)SO₂—; or —COO—;
Q represents lower alkylene; lower alkenylene;
M represents aryl-O(CH₂)ᵥR⁸; heteroaryl-O(CH₂)ᵥR⁸; aryl-O(CH₂)ᵥO(CH₂)_{w}R⁸; heteroaryl-(CH₂)ᵥO(CH₂)_{w}R⁸; aryl-OCH₂CH(R⁷)CH₂R⁵; heteroaryl-OCH₂CH(R⁷)CH₂R⁵;
L represents —R³; —COR³; —COOR³; —CONR²R³; —SO₂R³; —SO₂NR²R³;
—COCH(Aryl)₂;
K represents —H; —CH₂OR³; —CH₂NR²R³; —CH₂NR²COR³; —CH₂NR²SO₂R³; —CO₂R³; —CH₂OCONR²R³; —CONR²R³; —CH₂NR²CONR²R³; —CH₂SO₂NR²R³; —CH₂SR³; —CH₂SOR³; —CH₂SO₂R³;
R¹ represents hydrogen; lower alkyl; lower alkenyl; lower alkinyl; cycloalkyl; aryl; cycloalkyl-lower alkyl;
R² and R²′ independently represent hydrogen; lower alkyl; lower alkenyl; cycloalkyl; cycloalkyl-lower alkyl;
R³ represents hydrogen; lower alkyl; lower alkenyl; cycloalkyl; aryl; heteroaryl; heterocyclyl; cycloalkyl-lower alkyl; aryl-lower alkyl; heteroaryl-lower alkyl; heterocyclyl-lower alkyl; aryloxy-lower alkyl; heteroaryloxy-lower alkyl, whereby these groups may be unsubstituted or mono-, di- or trisubstituted with hydroxy, —OCOR², —COOR², lower alkoxy, cyano, —CONR²R²′, —CO-morpholin-4-yl, —CO-((4-loweralkyl)piperazin-1-yl), —NH(NH)NH², —NR⁴R⁴′ or lower alkyl, with the proviso that a carbon atom is attached at the most to one heteroatom in case this carbon atom is sp3-hybridized;
R⁴ and R⁴′ independently represent hydrogen; lower alkyl; cycloalkyl; cycloalkyl-lower alkyl; hydroxy-lower alkyl; —COOR²; —CONH₂;
R⁵ represents —OH, lower alkoxy, —OCOR², —COOR², —NR²R^{2′}, —OCONR²R²′, —NCONR²R²′, cyano, —CONR²R²′, SO₃H, —SONR²R²′, —CO-morpholin-4-yl, —CO-((4-loweralkyl)piperazin-1-yl), —NH(NH)NH₂, —NR⁴R⁴′, with the proviso that a carbon atom is attached at the most to one heteroatom in case this carbon atom is sp3-hybridized;
R⁶ represents hydrogen; lower alkyl; lower alkoxy, whereby these groups may be unsubstituted or monosubstituted with hydroxy, —CONH₂,
—COOH, imidazoyl, —NH₂, —CN, —NH(NH)NH₂;
R⁷ represents —OH, OR²; OCOR²; OCOOR²; or R⁶ and R⁵ form together with the carbon atoms to which they are attached a 1,3-dioxolane ring which is substituted in position 2 with R² and R²′;
or R⁶ and R⁵ form together with the carbon atoms to which they are attached a 1,3-dioxolan-2-one ring;
R⁸ represents lower alkoxy;
p is the integer . 2, 3 or 4;
r is the integer 1, 2, 3, 4, 5, or 6;
s is the integer 1, 2, 3, 4, or 5;
t is the integer 1, 2, 3, or 4;
u is the integer 1, 2, or 3;
v is the integer 1, 2,3, or 4;
w is the integer 1 or 2;
in any form, including optically pure enantiomers, mixtures of enantiomers such as racemates, diastereomers, mixtures of diastereomers, diastereomeric racemates, mixtures of diastereomeric racemates, and the meso-form; as well as free or pharmaceutically acceptable salts, solvent complexes and morphological forms.

3. Compounds according to claim 1 wherein
X represents —CH₂CH₂—.

4. Compounds according to claim 1 wherein
T represents —CONR¹—;
Q represents methylene;
M represents aryl-O(CH₂)ᵥR⁸; heteroaryl-O(CH₂)ᵥR⁸; aryl-OCH₂CH(R⁷)CH₂R⁵; heteroaryl-OCH₂CH(R⁷)CH₂R⁵.

5. Compounds according to claim 1 wherein
R¹ represents cycloalkyl;
R⁸ represents lower alkoxy
v represents 3.

6. Compounds according to claim 1 wherein
W represents a 1,4-disubstituted phenyl.

7. Compounds according to claim 1 wherein
U is a mono-, di-, or trisubstituted phenyl whereby the substituents are halogen; lower alkyl or lower alkoxy.

8. Compounds according to claim 1 wherein
U is a mono-, di-, or trisubstituted phenyl whereby the substituents are selected from fluorine and chlorine.

9. Compounds according to claim 1 wherein
V represents -A-(CH₂)ₛ—.

10. Compounds according to claim 1 wherein
A represents —O—, and
s represents 3.

11. The compounds according to claim 1 selected from the group consisting of
(rac.)-(1R *,5S*)-3-{4-[3-(2-chloro-3,6-difluorophenoxy)propyl]phenyl}-8-aza-bicyclo[3.2.1]oct-2-ene-2-carboxylic acid cyclopropyl-[2-(3-methoxypropoxy)-3-methylpyridin-4-ylmethyl]amide and pure enantiomers thereof, in free or pharmaceutically acceptable salt form.

12. A pharmaceutical composition comprising at least one five-membered heteroaryl derivative according to claim 1 in combination or association with a pharmaceutically acceptable carrier materials or adjuvants.

13. (canceled)

14. (canceled)

15. A method for the treatment or prophylaxis of diseases which are related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system, comprising the administration to a patient in need of such treatment or prophylaxis a pharmaceutically active amount of a five-membered heteroaryl derivative according to claim 1.

16. The method according to claim 15 wherein the five-membered heteroaryl derivative is (rac.)-(1R*,5S*)-3-{4-[3-(2-chloro-3,6-difluorophenoxy)propyl]phenyl}-8-aza-bicyclo[3.2.1]oct-2-ene-2-carboxylic acid cyclopropyl-[2-(3-methoxypropoxy)-3-methylpyridin-4-ylmethyl]amide, or a single enantiomer thereof, in free or pharmaceutically acceptable salt form.
